(19) United States
(12) Patent Application Publication
(10) Pub. No.: US 2020/0123103 A1
(43) Pub. Date: Apr. 23, 2020

(54) METHOD FOR THE SYNTHESIS OF AN N-ACYL COMPOUND WITHOUT THE USE OF ORGANIC SOLVENT OR ACID CHLORIDE

(71) Applicant: SOCIETE D'EXPLOITATION DE PRODUITS POUR LES INDUSTRIES CHIMIQUES SEPPIC, PARIS CEDEX 07 (FR)

(72) Inventors: Georges Manuel DA COSTA, SAIX (FR); Jérôme GUILBOT, CASTRES (FR); Eric PUJOL, PARIS CEDEX 07 (FR); Virginie POMMERY, Garrigues (FR); Fabrice SAUNAL, PARIS CEDEX 07 (FR)

(21) Appl. No.: 16/611,752

(22) PCT Filed: Apr. 27, 2018
(86) PCT No.: PCT/FR2018/051072

§ 371 Date: Nov. 7, 2019

(30) Foreign Application Priority Data
May 12, 2017 (FR) 1754188

(51) Int. Cl. C07D 207/16; C07D 403/12; A61K 8/49; A61K 31/403; A61K 31/401; A61K 9/00

(52) U.S. Cl. CPC C07D 207/16; C07D 403/12; A61K 8/4913; A61K 2800/59; A61K 31/401; A61K 9/0014; A61K 2800/10; A61K 31/403

(57) ABSTRACT

Disclosed is a method for the synthesis of N-acyl proline or N-acyl 3-hydroxy proline by way of the amide formation of a fatty acid at a high temperature, an intermediate composition of the method, and a cosmetic composition containing same.

The present invention relates to a novel process for the preparation of N-acylated derivatives of proline, not involving the use of solvent or of acid chlorides, and exhibiting a high degree of conversion of the starting materials employed. The present invention also relates to the use of the acylated products obtained for preparing cosmetic or pharmaceutical compositions for topical use or industrial detergency compositions.

N-Acylated derivatives of amino acids, also known as LipoAmino Acids (LAA), are anionic surfactants which are formed by a polar head originating from a residue of at least one amino acid, or else from a residue of (oligo)peptides or else from residues of partial or complete hydrolyzates of proteins, and by a hydrocarbon chain of lipophilic nature, originating from acid chlorides or methyl esters, which are themselves derived from oleochemistry.

These N-acylated derivatives of amino acids, of (oligo)peptides or else of partial or total hydrolyzates of proteins are commonly used first of all as ingredients which contribute foaming and cleansing properties for the preparation of cosmetic compositions, such as, for example, shower gels or shampoos, or else as ingredients which contribute biological properties for the preparation of cosmetic compositions intended to prevent or correct unsightly skin effects; said biological properties are, for example, antiaging, slimming, firming, depigmenting or pro-pigmenting properties.

The preparation process commonly employed for the preparation of such N-acylated derivatives of amino acids, of (oligo)peptides or else of partial or total hydrolyzates of proteins is known to a person skilled in the art under the name of Schotten-Baumann reaction.

Such a process is disclosed, for example, in the U.S. Pat. Nos. 2,463,779 and 6,703,517, in the publication J. Am. Oil Chem. Soc., 78 (1956), 172, and in the international patent applications published under the numbers WO 92/21318 and WO 94/26694.

This acylation process comprises a preliminary stage of salification of the amino acid, followed by a stage of acylation of the amino acid salt with an acid chloride, and then acidification of the N-acylated salt obtained.

The first stage (see Scheme 1) consists in neutralizing the amino acid, dissolved beforehand in water or in a mixture of water and of an organic cosolvent, with an inorganic base, usually aqueous sodium hydroxide or potassium hydroxide solution. The carboxyl group is then in an ionized form, thus making possible better solubility of the amino acid in water. The pH of this aqueous solution lies between 9.0 and 12.0, which makes it possible to ensure that the amine functional group of the amino acid is not protonated.

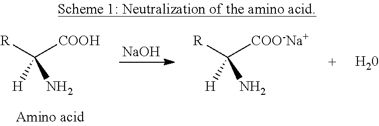

The second stage (see Scheme 2) is the acylation stage proper. At this stage, the acid chloride is gradually added to the neutralized solution of amino acid, at ambient temperature. The nucleophilic amine functional group attacks the electrophilic carbon of the carbonyl functional group. This results in the formation of an amide bond between the two starting substrates and also in the formation of hydrochloric acid. This acid is directly neutralized in situ by gradual addition of an inorganic base (regulation of the pH in the vicinity of 10.0).

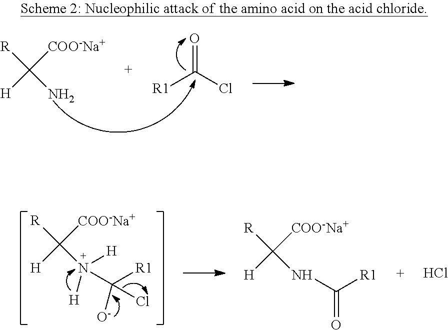

At this stage, the side reaction of hydrolysis of the acid chloride to give a soap (see Scheme 3) is also possible. It must, however, be minimized so as to achieve a satisfactory conversion of the amino acid to its N-acylated derivative and to efficiently isolate them, since an excessively high content of soap can bring about phase separation of the reaction medium and/or odor or toxicity problems (C₈ and C_{11′} chains, for example).

The two main reaction parameters which make it possible to control the formation of soap are:
i)—the stirring speed during the reaction phase, optimization of which makes possible an improvement in the contact surface area of the chloride with the medium, and
ii)—the optional addition, during the stage of dissolution of the amino acid, of an acylation cosolvent, such as, for example, acetone, methyl ethyl ketone, isopropanol or glycols.

This addition of cosolvent makes it possible to improve the affinity of the acid chloride for the reaction medium. In such a case, the acylation cosolvent must be astutely chosen so as to avoid or minimize the formation of new side products resulting from the reaction between this same cosolvent and the acid chloride, such as, for example, byproducts originating from esterification side reactions.

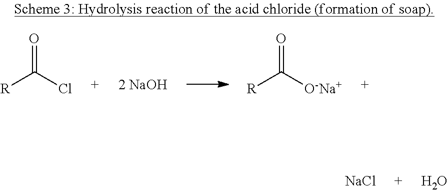

The final stage consists of a finishing stage, of placing in form the N-acylated derivative formed, and two alternatives are possible:
i)—The first consists in adjusting the value of the pH of the reaction medium obtained to the vicinity of 7. The N-acylated derivative is isolated as is in solution, without any additional purification, and comprises the acylation salts, the unreacted amino acids and the optional cosolvent. It is thus in a salified form, and more precisely a carboxylate form, in aqueous solution and with a purity generally of less than 50%.

ii)—The second consists in precipitating the N-acylated derivative by acidifying the reaction mixture to a pH value in the region of 2 and in then performing several filtration and washing operations, concluding with a final drying of the medium obtained. This procedure thus makes it possible to remove all the salts generated during the acylation reaction, the optional acylation cosolvent and all the unreacted amino acids. In this case, the N-acylated derivative is in a non-salified form, with a carboxylic acid functional group, and a solid form, more particularly a pulverulent form, and with a purity of greater than 80%.

The Schotten-Baumann process described above exhibits the advantage of carrying out N-acylation reactions with rapid kinetics, due to the very high reactivity of the acid chlorides toward nucleophilic compounds and functional groups (for example, the amine functional group), without a substantial supply of energy, such as, for example, heat energy, in a solvent medium predominantly formed of water, with high yields.

In return, this process has the disadvantages of carrying out the reactions in dilute media, which then reduces their productivity; of using acid chlorides as starting materials, obtained after reactions involving thionyl chloride or phosphorus trichloride as reagents, which are known for their hazardousness; of using organic cosolvents which are difficult to recycle; and of producing large amounts of inorganic salts, thus demanding treatment of the aqueous wash liquors.

The search for processes for the preparation of N-acylated derivatives of amino acids, alternative to those employing the Schotten-Baumann reaction, which are more environmentally friendly, is thus a relevant technical problem in the technical field.

Among the technical solutions, processes are known for the preparation of N-acylated derivatives of amino acids which consist in carrying out a direct amidation reaction of the amino acid by reaction with a fatty acid, or else in carrying out a transamidation reaction of the amino acid by reaction with a fatty acid ester, such as, for example, a methyl ester or an ethyl ester.

The United States patent published under the number U.S. Pat. No. 3,836,551 describes a process for the preparation of an N-acylated derivative of an amino acid by the reaction of a compound carrying a carboxylic acid functional group, or one of its esters, with an amino acid comprising at least three carbon atoms in the presence of an equimolar amount of a base, at a temperature of between 100° C. and 250° C.

The publication “High temperature reactions of fats with amino acids” (J. Am. Oil Chem. Soc., 1975, 52(5), pages 144-147) describes reactions carried out at high temperature (150° C. to 200° C.) of methyl esters (such as, for example, methyl myristate or methyl stearate) with various amino acids. However, these reactions are accompanied by decarboxylation and then amidation reactions, to result in the formation of fatty amides exhibiting the side chain of the amino acid, and thus in structures which are not those desired.

The German patent application published under the number DE 4 408 957 A1 describes the preparation of N-acyl sarcosinates from methyl esters, by introducing an intermediate stage of suspending sodium sarcosinate with a dispersant in the mixture of catalyst, more particularly sodium methoxide, and of solvent, more particularly methanol.

The United States patent published under the number U.S. Pat. No. 5,710,295 describes a process for the preparation of N-acyl amino acids by reaction of a fatty acid carrying a carboxylic functional group and of an amino acid at a temperature of between 100° C. and 200° C., in the presence of a mixture of water and of solvent, and more particularly of water and of isopropanol. However, the use of a solvent comprising an alcohol functional group makes it probable that an ester will be formed as undesired byproduct.

The international patent applications published under the numbers WO 2013/014238 and WO 2014/008103 disclose a process for the preparation of N-acyl amino acids by reaction between an amino acid and an alkyl ester, such as a methyl ester, of a fatty acid comprising from 8 to 22 carbon atoms in the presence of a polyol, such as glycerol or propylene glycol, in particular in the presence of a metal oxide, such as calcium oxide, or in the presence of sodium methoxide. Such a process makes it possible to achieve sparingly colored reaction products, but it is carried out in the presence of solvents and of metallic entities which must be subsequently removed. Furthermore, the presence of polyols in the solvent medium makes it probable that undesired esters will be formed.

The United States patent application published under the number US 2006/0239952 describes a process for the preparation of N-acyl amino acids, such as lauroyl amino acids, by reaction between an amino acid, such as, for example, glycine, alanine or threonine, and an excess amount of lauric acid, in particular greater than 1.5 molar equivalents, and in the presence of an alkaline substance, resulting in a large amount of saline substances. This process makes it possible to achieve mixtures of N-acylated derivatives of amino acids, and in particular of peptide entities, such as sodium N-lauroyl gluconate, sodium N-lauroyl (glycylglycinate) and sodium N-lauroyl (glycylglycylglycinate). The mixtures thus achieved are used for the preparation of compositions for cleansing human skin.

Proline is an amino acid which is very frequently used in the preparation of N-acylated derivatives because said N-acylated derivatives of proline exhibit several possibilities of use, in particular in the cosmetics, dermocosmetics and human or veterinary pharmaceutical industries, such as, for example, their uses as agents for dissolving hydrophobic molecules in water, such as, for example, essential oils.

Such a prior art shows that the alternative solutions to the processes for the preparation of N-acylated derivatives of amino acids, and in particular N-acylated derivatives of proline, alternatives to those employing the Schotten-Baumann reaction, employing fatty acids or fatty acid esters of natural origin, are unsatisfactory because they always involve catalysts in particular of metallic origin and/or organic solvents, and consequently stages for their removal and/or recycling, and/or the employment of alkaline substances which generate saline entities in amounts such that they require post-treatment stages.

Such a prior art in particular does not make it possible to obtain N-acylated derivatives of proline according to a process employing fatty acids or fatty acid esters of natural origin, and characterized by a high degree of conversion of the starting materials, and particularly by the production of a low residual content by weight of fatty acid or of fatty ester, and/or of proline.

This is why, in the context of their research studies aimed at changing the existing process toward a process employing starting materials of plant origin, in which no hazardous organic solvent is used, which results in a high conversion of the fatty acids, these acids causing skin irritations, the inventors have developed the process which is a subject matter of the present invention.

According to a first aspect, a subject matter of the invention is a process for the preparation of a compound of formula (I) or of a mixture of compounds of formula (I):

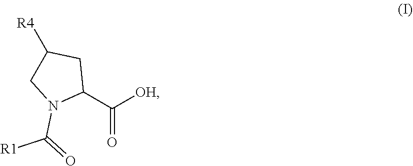

in which the R₁—C(═O)— group represents a saturated or unsaturated and linear or branched aliphatic radical comprising from 6 to 8 carbon atoms and R₄ represents a hydrogen atom or a hydroxyl radical, said process comprising:
a stage a) in which a fatty acid of formula (II):

R₁COOH (II)

in which R₁ is as defined in the formula (I), or a mixture of fatty acids of formula (II), is brought to a temperature of at least 100° C.;
a stage b) in which an aqueous solution of proline or of 3-hydroxyproline comprising, per 100% of its weight, from 30% to 60% by weight, more particularly from 40% a 60% by weight, of proline and from 40% to 70% by weight, more particularly from 40% to 60% by weight, of water is poured onto the heated fatty acid or the heated mixture of fatty acids resulting from stage a), in order to obtain a mixture (M₁) in which the molar ratio of the fatty acid of formula (II) or the mixture of fatty acids of formula (II) to proline or 3-hydroxyproline is greater than or equal to 0.4/1 and less than or equal to 0.8/1, more particularly greater than or equal to 0.5/1 and less than or equal to 0.8/1 and very particularly greater than or equal to 0.6/1 and less than or equal to 0.8/1;
a stage c) in which said mixture (M₁) obtained on conclusion of stage b) is maintained under mechanical stirring for at least 30 minutes and at a temperature of at least 100° C., in order to obtain an aqueous mixture (M₂) of the compound of formula (I) as defined above and of the compound of formula (III):

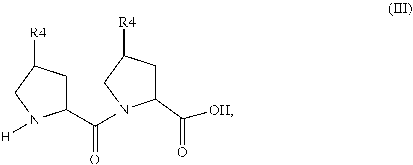

a stage d) of isolation of said compound of formula (I) or of said mixture of compounds of formula (I) from said mixture (M₂).

In stages a) and c) of the process as defined above, the term “at least 100° C.” means that the temperature can reach 220° C.

According to a specific embodiment of the process as defined above, the term “at least 100° C.” denotes, in stages a) and c), a temperature of between 130° C. and 220° C., more particularly between 140° C. and 200° C., for example between 140° C. and 180° C.

In stage c) of the process as defined above, the term “at least 30 minutes” denotes a duration which can reach 30 hours.

Stage d) of the process as defined above is generally carried out by liquid chromatography for separation purposes, by successive recrystallizations, by liquid-solid extraction, by liquid-liquid extraction, by extraction using an extraction fluid in its supercritical form, or by extraction with carbon dioxide in the supercritical state.

According to a specific embodiment of the process as defined above, the term “at least 30 minutes” denotes, in stage c), a duration of between 6 hours and 20 hours, more particularly between 8 hours and 20 hours.

According to another specific embodiment of the process as defined above, in the formula (II), the R₁—C(═O)— group represents a saturated or unsaturated and linear or branched radical comprising 7 or 8 carbon atoms and very particularly a saturated linear aliphatic radical comprising 7 or 8 carbon atoms.

According to another specific embodiment of the process as defined above, a proline solution is employed in stage b).

According to another specific embodiment of the process as defined above, the molar ratio of the fatty acid of formula (II) or the mixture of fatty acids of formula (II) to proline is greater than or equal to 0.6/1 and less than or equal to 0.8/1.

According to another specific embodiment of the process as defined above, the process comprises a stage e) of neutralization, and/or of filtration and/or of decolorization.

Another subject matter of the invention is the mixture (M₂), which is an intermediate of the process as defined above.

A subject matter of the invention is also a composition for topical use (C) comprising, per 100% of its weight:
from 0.1% to 40% by weight, more particularly from 0.1% to 20% by weight and more particularly still from 0.1% to 5% by weight of the mixture (M₂) as defined above, and
from 60% to 99.9%, more particularly from 80% to 99.9% and more particularly still from 95% to 99.9% by weight of a cosmetically acceptable medium.

The expression “for topical use” used in the definition of the cosmetic, dermocosmetic, dermopharmaceutical or pharmaceutical composition for topical use (C) as defined above means that said composition is employed by application to the skin, the hair, the scalp or the mucous membranes, whether it is a direct application in the case of a cosmetic, dermocosmetic, dermopharmaceutical or pharmaceutical composition or an indirect application, for example in the case of a body hygiene product in the form of a textile or paper wipe, or sanitary products intended to be in contact with the skin or the mucous membranes.

The expression “cosmetically acceptable” used in the definition of the cosmetic, dermocosmetic, dermopharmaceutical or pharmaceutical composition for topical use (C) means, according to the Council of the European Economic Community Directive No. 76/768/EEC of Jul. 27, 1976, amended by Directive No. 93/35/EEC of Jun. 14, 1993, any substance or preparation intended to be brought into contact with the various parts of the human body (epidermis, body hair and head hair system, nails, lips and genitals) or with the teeth and mucous membranes of the mouth, for the purpose, exclusively and mainly, of cleansing them, scenting them, modifying the appearance thereof and/or correcting body odors thereof and/or protecting them or keeping them in good condition. A cosmetically acceptable medium of these compositions which are a subject matter of the invention can conventionally contain water, one or more cosmetically acceptable organic solvents, or a mixture of water and of one or more organic solvents. The cosmetically acceptable solvents can more particularly be chosen from polyhydric alcohols, such as, for example, glycerol, diglycerol, glycerol oligomers, ethylene glycol, propylene glycol, hexylene glycol, diethylene glycol, xylitol, erythritol, sorbitol, or water-soluble alcohols.

The cosmetic, dermocosmetic, dermopharmaceutical or pharmaceutical composition for topical use (C) which is a subject matter of the present invention can be packaged in pressurized form in an aerosol device or in a device of “pump-action spray” type, in a device equipped with a perforated wall, for example a grill, or in a device equipped with a ball applicator (known as a “roll-on”). When it is packaged in small bottles, the composition (C) according to the invention as defined above can be applied in the form of fine droplets by means of mechanical pressurization devices or propellent gas devices. The propellants which can be combined with the composition (C) according to the invention include hydrofluorinated compounds, such as dichlorodifluoromethane, trichlorofluoromethane, difluoroethane, isobutane, butane and propane.

The cosmetic, dermocosmetic, dermopharmaceutical or pharmaceutical composition for topical use (C) as defined above can additionally comprise excipients and/or active principles generally employed in the field of formulations for topical use, in particular cosmetic, dermocosmetic, pharmaceutical or dermopharmaceutical formulations.

The cosmetic, dermocosmetic, dermopharmaceutical or pharmaceutical composition for topical use (C) which is a subject matter of the present invention and as defined above can additionally comprise one or more auxiliary compounds chosen from foaming and/or detergent surfactants, thickening and/or gelling surfactants, thickening and/or gelling agents, stabilizing agents, film-forming compounds, solvents and cosolvents, hydrotropic agents, plasticizing agents, opacifying agents, pearlescent agents, sequestrants, chelating agents, antioxidants, fragrances, essential oils, preserving agents, conditioning agents, deodorants, bleaching agents intended for bleaching bodily hair and the skin, active principles intended to contribute a treating and/or protective action with regard to the skin or the hair, sunscreens, inorganic fillers or pigments, particles which give a visual effect or which are intended for the encapsulation of active principles, exfoliant particles, texturing agents, optical brighteners and insect repellents.

The water-soluble antioxidants which can be combined with the cosmetic, dermocosmetic, dermopharmaceutical or pharmaceutical composition for topical use (C) according to the invention include ascorbic acid, glutathione, tartaric acid, oxalic acid and tetrasodium glutamate diacetate. The water-soluble sequestering agents which can be combined with the cosmetic, dermocosmetic, dermopharmaceutical or pharmaceutical composition for topical use (C) according to the invention include ethylenediaminetetraacetic acid (EDTA) salts, such as the sodium salt of EDTA, diethylenetriaminepentaacetic acid (DTPA) salts, such as the sodium salt of DTPA, and acetylglutamic acid (Dissolvine range).

The water-soluble dyes which can be combined with the cosmetic, dermocosmetic, dermopharmaceutical or pharmaceutical composition for topical use (C) according to the invention include caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, Ferric Ferrocyanide, Ultramarines, Acid Yellow 3/Yellow 10, Acid Blue 3 and Yellow 10.

The color-stabilizing water-soluble agents which can be combined with the cosmetic, dermocosmetic, dermopharmaceutical or pharmaceutical composition for topical use (C) according to the invention include tris(tetramethylhydroxypiperidinol) citrate, sodium benzotriazolyl butylphenol sulfonate and benzotriazolyl dodecyl p-cresol.

Mention may be made, as examples of foaming and/or detergent surfactants optionally present in the cosmetic, dermocosmetic, dermopharmaceutical or pharmaceutical composition for topical use (C) which is a subject matter of the present invention, of topically acceptable anionic, cationic, amphoteric or nonionic foaming and/or detergent surfactants generally used in this field of activity. Mention may be made, among the foaming and/or detergent anionic surfactants which can be combined with the cosmetic, dermocosmetic, dermopharmaceutical or pharmaceutical composition for topical use (C) which is a subject matter of the present invention, of alkali metal salts, alkaline earth metal salts, ammonium salts, amine salts, aminoalcohol salts of alkyl ether sulfates, of alkyl sulfates, of alkylamido ether sulfates, of alkylaryl polyether sulfates, of monoglyceride sulfates, of a-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkylsulfoacetates, of alkyl sarcosinates, of acylisethionates, of N-acyl taurates, of acyl lactylates, of N-acylated derivatives of amino acids, of N-acylated derivatives of peptides, of N-acylated derivatives of proteins or of fatty acids. Mention may be made, among the foaming and/or detergent amphoteric surfactants optionally present in the cosmetic, dermocosmetic, dermopharmaceutical or pharmaceutical composition for topical use (C) which is a subject matter of the present invention, of alkylbetaines, alkylamidobetaines, sultaines, alkylamidoalkylsulfobetaines, imidazoline derivatives, phosphobetaines, amphopolyacetates and amphopropionates. Mention may particularly be made, among the foaming and/or detergent cationic surfactants optionally present in the cosmetic, dermocosmetic, dermopharmaceutical or pharmaceutical composition for topical use (C) which is a subject matter of the present invention, of quaternary ammonium derivatives.

Mention may more particularly be made, among the foaming and/or detergent nonionic surfactants optionally present in the cosmetic, dermocosmetic, dermopharmaceutical or pharmaceutical composition for topical use (C) which is a subject matter of the present invention, of alkylpolyglycosides including a saturated or unsaturated and linear or branched aliphatic radical comprising from 8 to 12 carbon atoms; castor oil derivatives, polysorbates, coconut amides and N-alkylamines. Mention may be made, as examples of texturing agents optionally present in the cosmetic, dermocosmetic, dermopharmaceutical or pharmaceutical composition for topical use (C) which is a subject matter of the present invention, of N-acylated derivatives of amino acids, for example lauroyl lysine, sold under the name Aminohope™ LL, octenyl starch succinate, sold under the name Dryflo™, myristyl polyglucoside, sold under the name Montanov 14, cellulose fibers, cotton fibers, chitosan fibers, talc, sericite, mica and perlite.

Examples of active principles optionally present in the cosmetic, dermocosmetic, dermopharmaceutical or pharmaceutical composition for topical use (C) which is a subject matter of the present invention include:
vitamins and their derivatives, for example retinol (vitamin A) and its esters (for example retinyl palmitate), ascorbic acid (vitamin C) in salt form and its esters, sugar derivatives of ascorbic acid (for example ascorbyl glucoside), tocopherol (vitamin E) and its esters (for example tocopherol acetate), vitamin B3 or B10 (niacinamide and its derivatives);
compounds having a lightening or depigmenting action on the skin, for example Sepiwhite™ MSH, arbutin, kojic acid, hydroquinone, Vegewhite™, Gatuline™, Synerlight™ Biowhite™, Phytolight™, Dermalight™, Clariskin™, Melaslow™, Dermawhite™, Ethioline, Melarest™, Gigawhite™, Albatine™ and Lumiskin™;
compounds having a soothing action, such as Sepicalm™ S, allantoin and bisabolol;
anti-inflammatory agents;
compounds showing a moisturizing action, for example diglycerol, triglycerol, urea, hydroxyureas, glyceryl glucoside, diglyceryl glucoside, polyglyceryl glucosides, erythrityl glucoside, sorbityl glucoside, xylityl glucoside, the composition sold under the brand name Aquaxyl™ comprising xylityl glucoside, anhydroxylitol and xylitol;
compounds showing a slimming or lipolytic action, such as caffeine or its derivatives, Adiposlim™ and Adipoless™;
plant extracts rich in tannins, polyphenols and/or isoflavones, for example grape extracts, pine extracts, wine extracts, olive extracts; soybean extracts, for example Raffermine™; wheat extracts, for example Tensine™ or Gliadine™; terpene-rich plant extracts; extracts of freshwater or marine algae; marine extracts in general, such as corals;
compounds having an antimicrobial action or a purifying action, for example Lipacide™ C8G, Lipacide™ UG, Sepicontrol™ A5, Octopirox™ or Sensiva™ SC50;
compounds having an energizing or stimulating property, such as Physiogenyl™ panthenol and its derivatives, such as Sepicap™ MP;
antiaging active principles, such as Sepilift™ DPHP, Lipacide™ PVB, Sepivinol™ Sepivital™, Manoliva™, Phyto-Age™, Timecode™ or Survicode™;
antiphotoaging active principles;
active principles which increase the synthesis of the components of the extracellular matrix, for example collagen, elastins and glycosaminoglycans;
active principles which act favorably on chemical cell communication, such as cytokines, or physical cell communication, such as integrins;
active principles which create a feeling of “heating” on the skin, such as activators of cutaneous microcirculation (for example nicotinic acid derivatives) or products which create a feeling of “coolness” on the skin (for example menthol and its derivatives);
active agents which improve cutaneous microcirculation, for example venotonics; draining active principles; active principles having a decongestant purpose, for example extracts of Ginkgo biloba, ivy, horse chestnut, bamboo, ruscus, butcher's broom, Centella asiatica, fucus, rosemary or willow;
active principles which act as skin-tightening agents, for example hydrolyzates of plant proteins, hydrolyzates of marine origin, such as hydrolyzates of Laminaria extracts, hydrolyzates of fish cartilages, marine elastin, the product sold by SEPPIC under the brand name Sesaflash™, and collagen solutions;
agents for tanning or browning the skin, for example dihydroxyacetone, isatin, alloxan, ninhydrin, glyceraldehyde, mesotartaric aldehyde, glutaraldehyde or erythrulose.

Mention may be made, as examples of deodorants optionally present in the cosmetic, dermocosmetic, dermopharmaceutical or pharmaceutical composition for topical use (C) which is a subject matter of the present invention, of alkali metal silicates, zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol, 1,3-propanediol, salicylic acid, sodium bicarbonate, cyclodextrins, metal zeolites, Triclosan™, aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, or complexes of aluminum chlorohydrate and of glycol, such as the aluminum chlorohydrate and propylene glycol complex, the aluminum dichlorohydrate and propylene glycol complex, the aluminum sesquichlorohydrate and propylene glycol complex, the aluminum chlorohydrate and polyethylene glycol complex, the aluminum dichlorohydrate and polyethylene glycol complex or the aluminum sesquichlorohydrate and polyethylene glycol complex.

Mention may be made, as examples of thickening or gelling agents optionally present in the cosmetic, dermocosmetic, dermopharmaceutical or pharmaceutical composition for topical use (C) which is a subject matter of the present invention, of linear or branched or crosslinked polymers of polyelectrolyte type, such as the partially or completely salified acrylic acid homopolymer, the partially or completely salified methacrylic acid homopolymer, the partially or completely salified 2-methyl-[(1-oxo-2-propenyl)amino]-1-propanesulfonic acid (AMPS) homopolymer, copolymers of acrylic acid and of AMPS, copolymers of acrylamide and of AMPS, copolymers of vinylpyrrolidone and of AMPS, copolymers of AMPS and of 2-hydroxyethyl acrylate, copolymers of AMPS and of 2-hydroxyethyl methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of 2-hydroxyethyl acrylate, copolymers of acrylic or methacrylic acid and of 2-hydroxyethyl methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of 2-hydroxyethyl acrylate, terpolymers of acrylic or methacrylic acid, of AMPS and of 2-hydroxyethyl methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or methacrylic acid and of alkyl acrylates, the carbon chain of which comprises between 4 and 30 carbon atoms and more particularly between 10 and 30 carbon atoms, copolymers of AMPS and of alkyl acrylates, the carbon chain of which comprises between 4 and 30 carbon atoms and more particularly between 10 and 30 carbon atoms, linear, branched or crosslinked terpolymers of at least one monomer having a free, partially salified or completely salified strong acid functional group with at least one neutral monomer and at least one monomer of formula (VIII):

CH₂═C(R′₃)—C(═O)—[CH₂—CH₂—O]ₙ—R′₄ (VIII)

in which R′₃ represents a hydrogen atom or a methyl radical, R′₄ represents a linear or branched alkyl radical comprising from 8 to 30 carbon atoms and n represents a number greater than or equal to 1 and less than or equal to 50.

The linear or branched or crosslinked polymers of polyelectrolyte type which can be combined with the cosmetic, dermocosmetic, dermopharmaceutical or pharmaceutical composition for topical use (C) which is a subject matter of the present invention can be provided in the form of a solution, of an aqueous suspension, of a water-in-oil emulsion, of an oil-in-water emulsion, of a powder, for example the products sold under the names Simulgel™ EG, Simulgel™ EPG, Sepigel™ 305, Simulgel™ 600, Simulgel™ NS, Simulgel™ INS 100, Simulgel™ FL, Simulgel™ A, Simulgel™ SMS 88, Sepinov™ EMT 10, Sepiplus™ 400, Sepiplus™ 265, Sepiplus™ S, Sepimax™ Zen, Aristoflex™ AVC, Aristoflex™ AVS, Novemer™ EC-1, Novemer™ EC 2, Aristoflex™ HMB, Cosmedia™ SP, Flocare™ ET 25, Flocare™ ET 75, Flocare™ ET 26, Flocare™ ET 30, Flocare™ ET 58, Flocare™ PSD 30, Viscolam™ AT 64 and Viscolam™ AT 100; polysaccharides formed solely of monosaccharides, such as glucans or glucose homopolymers, glucomannoglucans, xyloglucans, galactomannans in which the degree of substitution (DS) of the D-galactose units on the D-mannose main chain is between 0 and 1 and more particularly between 1 and 0.25, such as galactomannans originating from cassia gum (DS=⅕), from locust bean gum (DS=¼), from tara gum (DS=⅓), from guar gum (DS=½) or from fenugreek gum (DS=1); polysaccharides formed of monosaccharide derivatives, such as galactan sulfates and more particularly carrageenans and agar, uronans and more particularly algins, alginates and pectins, heteropolymers of monosaccharides and of uronic acids and more particularly xanthan gum, gellan gum, gum arabic exudates and karaya gum exudates, or glucosaminoglycans; cellulose, cellulose derivatives, such as methylcellulose, ethylcellulose or hydroxypropyl cellulose, silicates, starch, hydrophilic starch derivatives or polyurethanes.

Mention may be made, as examples of oils optionally present in the cosmetic, dermocosmetic, dermopharmaceutical or pharmaceutical composition for topical use (C) which is a subject matter of the present invention, of mineral oils, such as liquid paraffin, liquid petrolatum, isoparaffins or white mineral oils; oils of animal origin, such as squalene or squalane; vegetable oils, such as phytosqualane, sweet almond oil, coconut oil, castor oil, jojoba oil, olive oil, rapeseed oil, peanut oil, sunflower oil, wheatgerm oil, corn germ oil, soybean oil, cottonseed oil, alfalfa oil, poppy oil, pumpkinseed oil, evening primrose oil, millet oil, barley oil, rye oil, safflower oil, candlenut oil, passionflower oil, hazelnut oil, palm oil, shea butter, apricot kernel oil, coriander seed oil, beechnut oil, calophyllum oil, sisymbrium oil, avocado oil, calendula oil, oils resulting from flowers or vegetables, or ethoxylated vegetable oils; synthetic oils, such as fatty acid esters, for example butyl myristate, propyl myristate, isopropyl myristate, cetyl myristate, isopropyl palmitate, octyl palmitate, butyl stearate, hexyldecyl stearate, isopropyl stearate, octyl stearate, isocetyl stearate, dodecyl oleate, hexyl laurate, propylene glycol dicaprylate, esters derived from lanolic acid, such as isopropyl lanolate or isocetyl lanolate, fatty acid monoglycerides, diglycerides and triglycerides, such as glycerol triheptanoate, alkyl benzoates, hydrogenated oils, poly(α-olefin)s, polyolefins, such as polyisobutene, synthetic isoalkanes, such as isohexadecane or isododecane, perfluorinated oils, silicone oils, such as polydimethylsiloxanes, polymethylphenylsiloxanes, silicones modified by amines, silicones modified by fatty acids, silicones modified by alcohols, silicones modified by alcohols and fatty acids, silicones modified by polyether groups, epoxy-modified silicones, silicones modified by fluorinated groups, cyclic silicones and silicones modified by alkyl groups. In the present patent application, the term “oils” is understood to mean compounds and/or mixtures of compounds which are insoluble in water, existing under a liquid appearance at a temperature of 25° C.

Mention may be made, as examples of waxes optionally present in the cosmetic, dermocosmetic, dermopharmaceutical or pharmaceutical composition for topical use (C) which is a subject matter of the present invention, of beeswax, carnauba wax, candelilla wax, ouricury wax, Japan wax, cork fiber wax, sugarcane wax, paraffin waxes, lignite waxes, microcrystalline waxes, lanolin wax, ozokerite, polyethylene wax, silicone waxes, vegetable waxes, fatty alcohols and fatty acids which are solid at ambient temperature, or glycerides which are solid at ambient temperature. In the present patent application, the term “waxes” is understood to mean compounds and/or mixtures of compounds which are insoluble in water, existing under a solid appearance at a temperature of greater than or equal to 45° C.

Mention may be made, as examples of nonionic emulsifying surfactants which can be combined with the cosmetic, dermocosmetic, dermopharmaceutical or pharmaceutical composition for topical use (C) which is a subject matter of the present invention, of esters of fatty acids and of sorbitol, such as the products sold under the names Montane™ 40, Montane™ 60, Montane™ 70, Montane™ 80 and Montane™ 85; the compositions comprising glyceryl stearate and ethoxylated stearic acid having between 5 mol and 150 mol of ethylene oxide, such as the composition comprising ethoxylated stearic acid having 135 mol of ethylene oxide and glyceryl stearate sold under the name Simulsol™ 165; mannitan esters; ethoxylated mannitan esters; sucrose esters; methyl glucoside esters; alkyl polyglycosides comprising a saturated or unsaturated and linear or branched aliphatic radical comprising from 14 to 36 carbon atoms, such as tetradecyl polyglucoside, hexadecyl polyglucoside, octadecyl polyglucoside, hexadecyl polyxyloside, octadecyl polyxyloside, eicosyl polyglucoside, dodecosyl polyglucoside, 2-octyldodecyl polyxyloside or 12-hydroxystearyl polyglucoside; or compositions of saturated or unsaturated and linear or branched fatty alcohols comprising from 14 to 36 carbon atoms and of alkyl polyglycosides as described above, for example the compositions sold under the brand names Montanov™ 68, Montanov™ 14, Montanov™ 82, Montanov™ 202, Montanov™ S, Montanov™ WO18, Montanov™ L, Fluidanov™ 20X and Easynov™.

Indicated as examples of agents for protecting against the ultraviolet rays of the sun optionally present in the cosmetic, dermocosmetic, dermopharmaceutical or pharmaceutical composition for topical use (C) which is a subject matter of the present invention are pigments, organic sunscreens and inorganic sunscreens.

Examples of pigments used as agent for protecting against the ultraviolet rays of the sun optionally present in the cosmetic, dermocosmetic, dermopharmaceutical or pharmaceutical composition for topical use (C) which is a subject matter of the present invention are titanium dioxide, brown iron oxides, yellow iron oxides, black iron oxides or red iron oxides, or else white or colored pearlescent pigments, such as titanium oxide-coated mica.

Examples of organic sunscreens used as agent for protecting against the ultraviolet rays of the sun optionally present in the cosmetic, dermocosmetic, dermopharmaceutical or pharmaceutical composition for topical use (C) which is a subject matter of the present invention are:
those of the family of the benzoic acid derivatives, such as para-aminobenzoic acids (PABAs), in particular monoglyceryl esters of PABA, ethyl esters of N,N-dipropoxy PABA, ethyl esters of N,N-diethoxy PABA, ethyl esters of N,N-dimethyl PABA, methyl esters of N,N-dimethyl PABA or butyl esters of N,N-dimethyl PABA;
those of the family of the anthranilic acid derivatives, such as homomenthyl N-acetylanthranilate;
those of the family of the salicylic acid derivatives, such as amyl salicylate, homomenthyl salicylate, ethylhexyl salicylate, phenyl salicylate, benzyl salicylate or p-isopropylphenyl salicylate;
those of the family of the cinnamaic acid derivatives, such as ethylhexyl cinnamate, ethyl 4-isopropylcinnamate, methyl 2,5-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate or mono(2-ethylhexanoyl)glyceryl di(para-methoxycinnamate);
those of the family of the benzophenone derivatives, such as 2,4-dihydroxybenzophenone, 2,2′-dihydroxy-4-methoxybenzophenone, 2,2,′4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-(n-octyloxy)benzophenone, 4-hydroxy-3-carboxybenzophenone, 3-(4′-methylbenzylidene)-D,L-camphor, 3-(benzylidene)-D,L-camphor, camphor benzalkonium methosulfate, urocanic acid or ethyl urocanate;
those of the family of the sulfonic acid derivatives, such as 2-phenylbenzimidazole-5-sulfonic acid and its salts; the family of the triazine derivatives, such as hydroxyphenyl triazine, ethylhexyloxyhydroxyphenyl-4-methoxyphenyltriazine, 2,4,6-trianilino-(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis(2-ethylhexyl) ester of benzoic acid, 2-phenyl-5-methylbenzoxazole, 2,2′-hydroxy-5-methylphenylbenzotriazole, 2-(2′-hydroxy-5′-(t-octyl)phenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; 2-(4-diethylamino-2-hydroxybenzoyl)benzoic acid hexyl ester, 2,4-bis{[4-(2-ethylhexyloxy)-2-hydroxy]phenyl}-6-(4-methoxyphenyl)-1,3,5-triazine, 2,4,6-tris[4-(2-ethylhexyloxycarbonyl)anilino]-1,3,5-triazine, 2-ethylhexyl dimethoxybenzylidene dioxoimidazolidine propionate, the family of the diphenylacrylate derivatives, such as 2-ethylhexyl 2-cyano-3,3-diphenyl-2-propenoate or ethyl 2-cyano-3,3-diphenyl-2-propenoate;
those of the family of the polysiloxanes, such as benzylidene siloxane malonate.

Examples of inorganic sunscreens used as agent for protecting against the ultraviolet rays of the sun optionally present in the cosmetic, dermocosmetic, dermopharmaceutical or pharmaceutical composition for topical use (C) which is a subject matter of the present invention are: titanium oxides, zinc oxides, cerium oxide, zirconium oxide, yellow, red or black iron oxides, and chromium oxides. These mineral screens may or may not be micronized, may or may not have undergone surface treatments and may be optionally presented in the forms of aqueous or oily predispersions.

The following examples illustrate the invention without, however, limiting it.

### EXAMPLE 1

### Preparation of a Composition (C) According to the Invention

62.6 grams of n-heptanoic acid, i.e. 0.8 molar equivalent, are introduced into a reactor equipped with a jacket comprising a heat-exchange fluid, with a mechanical stirring system equipped with a stirrer of propeller or anchor type, and with a water evacuation system. The temperature is brought to 150° C. and the stirring is started in order to homogenize the heptanoic acid for a period of 10 minutes.

69.54 grams of an aqueous proline solution, containing 50% by weight of proline, i.e. one molar equivalent of proline, are subsequently added gradually, and such a mixing is maintained for a period of time of 12 hours 30 minutes, at a temperature of 155° C., under a partial vacuum of 4×10⁴ Pa to 7×10⁴ Pa (400 to 700 mbar). The reaction medium obtained is cooled to a temperature of 90° C. and then emptied out in order to obtain the composition (C_{1A}), which is subsequently characterized analytically.

### EXAMPLE 2

### Preparation of a Composition (C_{1B}) According to the Invention

The procedure of the process described in example 1 is employed, the 0.8 molar equivalent of n-heptanoic acid being replaced with 0.8 molar equivalent of n-octanoic acid.

Such a mixing is then maintained for a period of time of 11 hours, at a temperature of 155° C., under a partial vacuum of 2×10⁴ Pa to 4×10⁴ Pa (200 to 400 mbar). The reaction medium obtained is cooled to a temperature of 90° C. and then emptied out in order to obtain the composition (C_{1B}), which is subsequently characterized analytically.

### COMPARATIVE EXAMPLE 1

### Preparation of a Comparative Composition (C′₁)

The procedure of the process described in example 1 is employed, the 0.8 molar equivalent of n-heptanoic acid being replaced with 0.8 molar equivalent of n-lauric acid.

Such a mixing is then maintained for a period of time of 6 hours 30 minutes, at a temperature of 155° C., under a partial vacuum of 3×10⁴ Pa (300 mbar). The reaction medium obtained is cooled to a temperature of 90° C. and then emptied out in order to obtain the comparative composition (C′₁), which is subsequently characterized analytically.

### COMPARATIVE EXAMPLE 2

### Preparation of a Comparative Composition (C′₂)

The procedure of the process described in example 1 is employed, the 0.8 molar equivalent of n-heptanoic acid being replaced with 0.8 molar equivalent of n-palmitic acid.

Such a mixing is then maintained for a period of time of 6 hours 30 minutes, at a temperature of 155° C., under a partial vacuum of 2×10⁴ Pa to 6×10⁴ Pa (200 to 600 mbar). The reaction medium obtained is cooled to a temperature of 90° C. and then emptied out in order to obtain the comparative composition (C′₂), which is subsequently characterized analytically.

### COMPARATIVE EXAMPLE 3

### Preparation of a Comparative Composition (C′₃)

The procedure of the process described in example 1 is employed, the 0.8 molar equivalent of n-heptanoic acid being replaced with 0.8 molar equivalent of n-octanoic acid and the one molar equivalent of proline being replaced with one molar equivalent of glycine.

No reaction is observed and the medium sets solid. The comparative composition (C′₃) thus consists of a mixture of octanoic acid and of glycine.

Characterization of the Compositions (C_{1A}) and (C_{1B}) According to the Invention and of the Comparative Compositions (C′₁), (C′₂) and (C′₃).

The compositions (C_{1A}) and (C_{1B}) according to the invention and the comparative compositions (C′₁), (C′₂) and (C′₃) obtained are characterized by the visual evaluation of the appearance, the measurement of the acid number and also the determination of the contents by weight of residual fatty acid, residual proline, diproline, N-acylated derivative of proline and N-acylated dipeptide derivative of proline by gas chromatography by means of a chromatograph comprising an injection system, a flame ionization detector provided with a data acquisition system and equipped with a CP Simdist Ultimetal column (100% methylsiloxane 10 m×0.53 mm ID, film thickness 0.53 μm), helium being used as carrier gas.

The conversion of the starting materials will be judged to be unsatisfactory if the content by weight of fatty acid in the composition resulting from the process for obtaining N-acylated derivatives of proline is greater than or equal to 10% per 100% by weight of the composition obtained. This is because, above such a content by weight, reactions of irritation of the skin, caused by the fatty acid, are liable to be encountered and to then render such a composition unsuitable for a cosmetic use.

The analytical characterizations of the compositions (C_{1A}) and (C_{1B}) according to the invention and the comparative compositions (C′₁), (C′₂) and (C′₃) obtained are recorded in the following tables 1 and 2:

TABLE 1

| | Composition | Composition |
|---|---|---|
| | (C_{1A}) | (C_{1B}) |
| Appearance at 25° C. (visual) | Viscous | Viscous |
| | liquid | liquid |
| Acid number (mg KOH/g) (NFT 60-204) | 142.8 | 124.0 |
| Heptanoic acid (% by weight) | 2.0% | — |
| Octanoic acid (% by weight) | — | 1.4% |
| Proline (% by weight) | n.d. | 0.4% |
| Proline-proline (% by weight) | 28.3% | 22.4% |
| N-Heptanoylproline (% by weight) | 67.5% | — |
| N-Octanoylproline (% by weight) | — | 55.6% |
| N-Heptanoylproline-proline (% by weight) | 2.2% | — |
| N-Octanoylproline-proline (% by weight) | — | 9.2% |

n.d.: not determined.

TABLE 2

| | Composition | Composition |
|---|---|---|
| | (C′₁) | (C′₂) |
| Appearance at 25° C. (visual) | Viscous | Viscous |
| | liquid | liquid |
| Acid number (mg KOH/g) (NFT 60-204) | 164.0 | 145.0 |
| Lauric acid (% by weight) | 30.4% | — |
| Palmitic acid (% by weight) | — | 45.2% |
| Proline (% by weight) | n.d. | n.d. |
| Proline-proline (% by weight) | 9.7% | 6.8% |
| N-Lauroylproline (% by weight) | 57.6% | — |
| N-Palmitoylproline (% by weight) | — | 46.7% |
| N-Lauroylproline-proline (% by weight) | 2.3% | — |
| N-Palmitoylproline-proline (% by weight) | — | 1.3% |

These characterizations demonstrate that the contents by weight of the residual fatty acids and of the compounds of formula (II) of the compositions (C_{1A}) and (C_{1B}) obtained by the implementation of the process according to the invention are markedly less than 10% by weight, whereas those of fatty acids of the comparative compositions (C′₁) and (C′₂) are, on the contrary, markedly greater than 10% by weight.

1. A process for the preparation of a compound of formula (I) or of a mixture of compounds of formula (I): 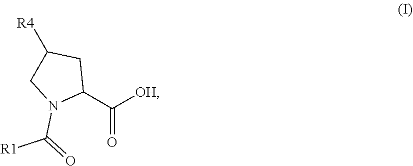
in which the R₁—C(═O)— group represents a saturated or unsaturated and linear or branched aliphatic radical comprising from 6 to 8 carbon atoms and R₄ represents a hydrogen atom or a hydroxyl radical, said process comprising:
a stage a) in which a fatty acid of formula (II):
R₁COOH (II)
in which R₁ is as defined in the formula (I), or a mixture of fatty acids of formula (II), is brought to a temperature of at least 100° C.;
a stage b) in which an aqueous solution of proline or of 3-hydroxyproline comprising, per 100% of a weight of the aqueous solution, from 30% to 60% by weight of proline and from 40% to 70% by weight of water is poured onto the heated fatty acid or the heated mixture of fatty acids resulting from stage a), in order to obtain a mixture (M₁) in which the molar ratio of the fatty acid of formula (II) or the mixture of fatty acids of formula (II) to proline or 3-hydroxyproline is greater than or equal to 0.4/1 and less than or equal to 0.8/1;
a stage c) in which said mixture (M₁) obtained on conclusion of stage b) is maintained under mechanical stirring for at least 30 minutes and at a temperature of at least 100° C., in order to obtain an aqueous mixture (M₂) of the compound of formula (I) as defined above and of the compound of formula (III): 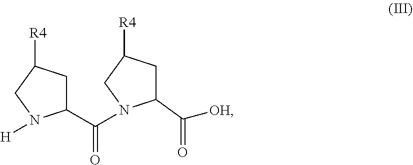
a stage d) of isolation of said compound of formula (I) or of said mixture of compounds of formula (I) from said mixture (M₂).

2. The process as defined in claim 1, wherein, in the formula (I), the R₁—C(═O)— group represents an n-heptanoyl radical or an n-octanoyl radical.

3. The process as defined in claim 1, wherein, in stage b), a proline solution is employed.

4. The process as defined in claim 3, wherein the molar ratio of the fatty acid of formula (II) or the mixture of fatty acids of formula (II) to proline is greater than or equal to 0.6/1 and less than or equal to 0.8/1.

5. A mixture (M₂), which is an intermediate of the process as defined in claim 1.

6. A mixture (M₂), which is an intermediate of the process as defined in claim 2.

7. A mixture (M₂), which is an intermediate of the process as defined in claim 3.

8. A mixture (M₂), which is an intermediate of the process as defined in claim 4.

9. A cosmetic, dermocosmetic, dermopharmaceutical or pharmaceutical composition for topical use (C) comprising, per 100% of a weight of the composition:
from 0.1% to 40% by weight of the mixture (M₂) as defined in claim 5, and
from 60% to 99.9% by weight of a cosmetically acceptable medium.

10. The process as defined in claim 2, wherein, in stage b), a proline solution is employed.

11. A cosmetic, dermocosmetic, dermopharmaceutical or pharmaceutical composition for topical use (C) comprising, per 100% of a weight of the composition:
from 0.1% to 40% by weight of the mixture (M₂) as defined in claim 6, and
from 60% to 99.9% by weight of a cosmetically acceptable medium.

12. A cosmetic, dermocosmetic, dermopharmaceutical or pharmaceutical composition for topical use (C) comprising, per 100% of a weight of the composition:
from 0.1% to 40% by weight of the mixture (M₂) as defined in claim 7, and
from 60% to 99.9% by weight of a cosmetically acceptable medium.

13. A cosmetic, dermocosmetic, dermopharmaceutical or pharmaceutical composition for topical use (C) comprising, per 100% of a weight of the composition:
from 0.1% to 40% by weight of the mixture (M₂) as defined in claim 8, and
from 60% to 99.9% by weight of a cosmetically acceptable medium.
